(19) United States
(12) Patent Application Publication
(10) Pub. No.: US 2021/0346433 A1
(43) Pub. Date: Nov. 11, 2021

(54) LET-7 PROMOTES ANTI-TUMOR ACTIVITY OF CD8 T CELLS AND MEMORY FORMATION IN VIVIO

(71) Applicant: University of Massachusetts, Boston, MA (US)

(72) Inventors: Leonid Pobezinsky, Leverett, MA (US); Elena Pobezinskaya, Leverett, MA (US)

(21) Appl. No.: 17/250,988

(22) PCT Filed: Oct. 29, 2019
(86) PCT No.: PCT/US2019/058524

§ 371 Date: Apr. 8, 2021

Related U.S. Application Data
(60) Provisional application No. 62/752,062, filed on Oct. 29, 2018.

(51) Int. Cl. A61K 35/17; C12N 5/0783; A61P 35/00; A61K 45/06; A61P 35/04

(52) U.S. Cl. CPC A61K 35/17; C12N 5/0636; A61P 35/00; C12N 2510/00; A61K 45/06; A61P 35/04; C12N 5/0638

(57) ABSTRACT

Disclosed herein are compositions and methods for enhancing T-cell activity by modulating a miRNA so as to improve T-cell therapies. Described herein is the discovery that miRNA (Iet7) regulates T cell responses (including both T-helpers and cytotoxic CD8 Lymphocyte (CTLs)). Described herein are compositions and methods to increase the cytotoxic activity of CTLs and improve cancer immunotherapies.

### PRIORITY APPLICATION

This application claims priority to U.S. Provisional Application Ser. No. 62/752,062, filed Oct. 29, 2018, the disclosure of which is incorporated herein in its entirety by reference.

### BACKGROUND OF THE INVENTION

The immune system provides the only known intrinsic mechanism that eliminates malignant cells from an organism. Cytotoxic CD8⁺ T Lymphocytes (CTLs), aided by T-helper cells, are the most potent killer-cells among all immuno-competent cell types involved in anti-tumor responses. Unfortunately, CTL-mediated immuno-surveillance is far from perfect. Malignant cells often escape the immune response by acquiring immunosuppressive properties or generating an immunosuppressive environment, making tumor-derived antigens less immunogenic than they might otherwise be.

### SUMMARY OF THE INVENTION

The invention improves cancer immunotherapies that are based on adoptive T cell transfer/improves T-cell therapies.

Described herein is the discovery that miRNA (let7) regulates T cell responses (including both T-helpers and cytotoxic CD8⁺ Lymphocyte (CTLs)). Described herein are compositions and methods to increase the cytotoxic activity of CTLs and improve cancer immunotherapies. For example, increasing let7 expression in CTLs results in an increase in activation of these cells in vivo. Thus, let7 levels can be manipulated in vitro in adoptive cell therapies, such therapy can result in enhanced CTL anti-tumor responses when these cells are injected back into patients.

One embodiment provides a composition comprising T cells with increased expression of let7 as compared to wild-type cells, wherein the T cells comprise decreased expression of PD1, Lag3, Tim3, CD160, and 2B4 and increased expression of CD62L, IL-7Ra, TCF7, CCR7, LEF1 and ID3 as compared to wild type T cells. In one embodiment, the T cells are cytotoxic T lymphocyte cells.

Another embodiment provides a method to treat cancer comprising administering to a subject in need thereof an effective amount of T cells with increased expression of let7 as compared to wild-type T cells. In one embodiment, the administered T cells further comprise decreased expression of PD1, Lag3, Tim3, CD160, and 2B4 and increased expression of CD62L, IL-7Ra, TCF7, CCR7, LEF1 and ID3 as compared to wild type T cells. In another embodiment, the T cells are cytotoxic T lymphocyte cells. In one embodiment, the cancer is melanoma or lymphoma.

Another embodiment provides a method to increase cytotoxic activity of cytotoxic T lymphocyte (CTL) cells in vivo comprising increasing the level of let7 expression in said CTL cells.

In some embodiments, let7 expression is increased by increasing let7 copy number or by increasing expression by inserting a strong promoter in front of let7 coding region.

Some aspects provide administering at least one or more additional cancer treatments, such as chemotherapy, radiation and/or immunotherapy.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-C: Let-7 Tg CTLs control tumor growth. Schematic representation of the experiment. WT mice were given B16 tumor cells s.c. Tumors were measured every 2-3 days starting on day 8 (A). Tumor volumes (B). Kaplan-Meier survival graph (C).

FIG. 2: Let-7 expression inversely correlates with exhausted phenotype of effector T cells. Surface staining of PD-1 and Tim-3 on in vitro generated CTLs with different levels of let-7.

FIGS. 3A-B: Let-7 expression correlates with memory phenotype of effector T cells. Surface staining of CD62L, IL-7Ra (CD127) and CD44 on in vitro generated CTLs (5 days) with different levels of let-7. Numbers represent MFI of the staining; gray dotted line is isotype control (A). Survival of in vitro generated CTLs from indicated mice upon cytokine (IL-2) withdrawal on the 5th day of culture (B). Experiments were repeated at least two times. ***P<0.001, *P<0.05. Stud. two-tailed t-test.

FIGS. 4A-B: Let-7 Tg CTLs control tumor growth. Schematic representation of the experiment. WT mice were given B16 tumor cells i.v. followed by CTL injection on day 2. A group of 3 mice that received Lin28Tg CTLs was also given anti-PDL1 on the indicated days (A). Photograph of lungs from mice on day 14 after tumor injection (B).

### DETAILED DESCRIPTION OF THE INVENTION

Disclosed herein are compositions and methods for enhancing T-cell activity by modulating a miRNA so as improve T-cell therapies. Further disclosed herein is the discovery of compositions and methods that can improve cancer immunotherapies; in particular those that are based on adoptive T cell transfer (e.g., compatible with new and existing methods for T-cell therapies).

For the purposes of clarity and a concise description, features can be described herein as part of the same or separate embodiments; however, it will be appreciated that the scope of the invention may include embodiments having combinations of all or some of the features described.

The terminology used herein is for the purpose of describing particular embodiments only and is not intended to be limiting of the invention.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of”

As used herein, the terms “including,” “includes,” “having,” “has,” “with,” or variants thereof, are intended to be inclusive similar to the term “comprising.”

As used herein, the term “about” means plus or minus 10% of the indicated value. For example, about 100 means from 90 to 110.

As used herein, the term “subject” refers to any animal (e.g., mammals, birds, reptiles, amphibians, fish), including, but not limited to, humans, non-human primates, rodents, and the like, which is to be the recipient of a particular treatment. Typically, the terms “subject” and “patient” may be used interchangeably herein in reference to a subject.

As used herein, the term “administering” refers to providing a therapeutically effective amount of a chemical or biological compound (such as a cell, protein or oligonucleotide) or pharmaceutical composition to a subject. The chemical or biological compound of the present invention can be administered alone, but may be administered with other compounds, excipients, fillers, binders, carriers or other vehicles selected based upon the chosen route of administration and standard pharmaceutical practice. Administration may be by way of carriers or vehicles, such as injectable solutions, including sterile aqueous or non-aqueous solutions, or saline solutions; creams; lotions; capsules; tablets; granules; pellets; powders; suspensions, emulsions, or microemulsions; patches; micelles; liposomes; vesicles; implants, including microimplants; eye drops; ear drops; sprays, including nasal sprays; other proteins and peptides; synthetic polymers; microspheres; nanoparticles; and the like.

The chemical or biological compound or pharmaceutical composition of the present invention may also be included, or packaged, with other non-toxic compounds, such as pharmaceutically acceptable carriers, excipients, binders and fillers including, but not limited to, glucose, lactose, gum acacia, gelatin, mannitol, xanthan gum, locust bean gum, galactose, oligosaccharides and/or polysaccharides, starch paste, magnesium trisilicate, talc, corn starch, starch fragments, keratin, colloidal silica, potato starch, urea, dextrans, dextrins, and the like. Moreover, the packaging material may be biologically inert or lack bioactivity, such as plastic polymers, silicone, etc. and may be processed internally by the subject without affecting the effectiveness of the agent packaged and/or delivered therewith.

The term “effective amount,” as applied to the compound(s), biologics and pharmaceutical compositions described herein, means the quantity necessary to render the desired therapeutic result. For example, an effective amount is a level effective to treat, cure, or alleviate the symptoms of a disorder for which the therapeutic compound, biologic or composition is being administered. Amounts effective for the particular therapeutic goal sought will depend upon a variety of factors including the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage can occur depending upon the condition of the subject being treated, and the physician or other individual administering treatment will, in any event, determine the appropriate dose for an individual patient.

As used herein, “disorder” refers to a disorder, disease or condition, or other departure from healthy or normal biological activity, and the terms can be used interchangeably. The terms would refer to any condition that impairs normal function. The condition may be caused by sporadic or heritable genetic abnormalities. The condition may also be caused by non-genetic abnormalities. The condition may also be caused by injuries to a subject from environmental factors, such as, but not limited to, cutting, crushing, burning, piercing, stretching, shearing, injecting, or otherwise modifying a subject's cell(s), tissue(s), organ(s), system(s), or the like.

As used herein, “treatment” or “treating” refers to arresting or inhibiting, or attempting to arrest or inhibit, the development or progression of a disorder and/or causing, or attempting to cause, the reduction, suppression, regression, or remission of a disorder and/or a symptom thereof. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms. Additionally, treatment can be applied to a subject or to a cell culture.

microRNA

A micro RNA (abbreviated miRNA) is a small non-coding RNA molecule (containing about 22 nucleotides) found in plants, animals and some viruses, that functions in RNA silencing and post-transcriptional regulation of gene expression.

Encoded by eukaryotic nuclear DNA in plants and animals and by viral DNA in certain viruses whose genome is based on DNA, miRNAs function via base-pairing with complementary sequences within mRNA molecules. As a result, these mRNA molecules are silenced, by one or more of the following processes: cleavage of the mRNA strand into two pieces; destabilization of the mRNA through shortening of its poly(A) tail; and less efficient translation of the mRNA into proteins by ribosomes.

miRNAs resemble the small interfering RNAs (siRNAs) of the RNA interference (RNAi) pathway, except miRNAs derive from regions of RNA transcripts that fold back on themselves to form short hairpins, whereas siRNAs derive from longer regions of double-stranded RNA.

The first miRNA was discovered in the early 1990s. However, miRNAs were not recognized as a distinct class of biological regulators until the early 2000s. miRNA research revealed different sets of miRNAs expressed in different cell types and tissues and multiple roles for miRNAs in plant and animal development and in many other biological processes. Aberrant miRNA expression is implicated in disease states. MiRNA-based therapies are under investigation.

The mature miRNA is part of an active RNA-induced silencing complex (RISC) containing Dicer and many associated proteins. RISC is also known as a microRNA ribonucleoprotein complex (miRNP); RISC with incorporated miRNA is sometimes referred to as “miRISC.”

Dicer processing of the pre-miRNA is thought to be coupled with unwinding of the duplex. Generally, only one strand is incorporated into the miRISC, selected on the basis of its thermodynamic instability and weaker base-pairing on the 5′ end relative to the other strand. The position of the stem-loop may also influence strand choice. The other strand, called the passenger strand due to its lower levels in the steady state, is denoted with an asterisk (*) and is normally degraded. In some cases, both strands of the duplex are viable and become functional miRNA that target different mRNA populations.

Gene silencing may occur either via mRNA degradation or preventing mRNA from being translated. For example, miR16 contains a sequence complementary to the AU-rich element found in the 3′UTR of many unstable mRNAs, such as TNF alpha or GM-CSF. It has been demonstrated that given complete complementarity between the miRNA and target mRNA sequence, Ago2 can cleave the mRNA and lead to direct mRNA degradation. Absent complementarity, silencing is achieved by preventing translation. The relation of miRNA and its target mRNA(s) can be based on the simple negative regulation of a target mRNA, but it seems that a common scenario is the use of a “coherent feed-forward loop,” “mutual negative feedback loop” (also termed double negative loop) and “positive feedback/feed-forward loop” Some miRNAs work as buffers of random gene expression changes arising due to stochastic events in transcription, translation and protein stability. Such regulation is typically achieved by the virtue of negative feedback loops or incoherent feed-forward loop uncoupling protein output from mRNA transcription.

miRNA Let7

The Let-7 microRNA precursor was identified from a study of developmental timing in C. elegans, (Rougvie, A E (2001) Nature Reviews Genetics 2 (9): 690-701) and was later shown to be part of a much larger class of non-coding RNAs termed microRNAs (Ambros, V (2001) Cell 107 (7): 823-826). miR-98 microRNA precursor from human is a let-7 family member. Let-7 miRNAs have now been predicted or experimentally confirmed in a wide range of species (MIPF0000002). miRNAs are initially transcribed in long transcripts (up to several hundred nucleotides) called primary miRNAs (pri-miRNAs), which are processed in the nucleus by Drosha and Pasha to hairpin structures of about ˜70 nucleotide. These precursors (pre-miRNAs) are exported to the cytoplasm by exportin5, where they are subsequently processed by the enzyme Dicer to a ˜22 nucleotide mature miRNA. The involvement of Dicer in miRNA processing demonstrates a relationship with the phenomenon of RNA interference.

In the human genome, the cluster let-7a-1/let-7f-1/let-7d is inside the region B at 9q22.3, with the defining marker D9S280-D9S1809. One minimal LOH (loss of heterozygosity) region, between loci D11S1345-D11S1316, contains the cluster miR-125b1/let-7a-2/miR-100. The cluster miR-99a/let-7c/miR-125b-2 is in a 21p11.1 region of HD (homozygous deletions). The cluster let-7g/miR-135-1 is in region 3 at 3p21.1-p21.2 (Calin et al. (2003) PNAS 101 (9): 2999-3004).

The sequences, expression timing, as well as genomic clustering of the vertebrate miRNAs members are all conserved across species (Rodriguez A.; et al. (2004) Genome Res. 14 (10A): 1902-1910.) The direct role of let-7 family in vertebrate development has not been clearly shown as in less complex organisms, yet the expression pattern of let-7 family is indeed temporal during developmental processes (Kloosterman W. P. and Plasterk R. H. (2006) Dev. Cell 11 (4): 441-450). Let-7 sequences (both human and mouse) and accession # are provided below (modifications of these sequences are included as part of the invention, including use of non-natural nucleotides and those sequences which are at least about 95% identical):

>hsa-let-7a-5p MIMAT0000062
(SEQ ID NO: 1)
UGAGGUAGUAGGUUGUAUAGUU
>hsa-let-7b-5p MIMAT0000063
(SEQ ID NO: 2)
UGAGGUAGUAGGUUGUGUGGUU
>hsa-let-7c-5p MIMAT0000064
(SEQ ID NO: 3)
UGAGGUAGUAGGUUGUAUGGUU
>hsa-let-7d-5p MIMAT0000065
(SEQ ID NO: 4)
AGAGGUAGUAGGUUGCAUAGUU
>hsa-let-7e-5p MIMAT0000066
(SEQ ID NO: 5)
UGAGGUAGGAGGUUGUAUAGUU
>hsa-let-7f-5p MIMAT0000067
(SEQ ID NO: 6)
UGAGGUAGUAGAUUGUAUAGUU
>hsa-let-7g-5p MIMAT0000414
(SEQ ID NO: 7)
UGAGGUAGUAGUUUGUACAGUU
>hsa-let-7i-5p MIMAT0000415
(SEQ ID NO: 8)
UGAGGUAGUAGUUUGUGCUGUU
>hsa-miR-98-5p MIMAT0000096
(SEQ ID NO: 9)
UGAGGUAGUAAGUUGUAUUGUU
>mmu-let-7a-5p MIMAT0000521
(SEQ ID NO: 10)
UGAGGUAGUAGGUUGUAUAGUU
>mmu-let-7b-5p MIMAT0000522
(SEQ ID NO: 11)
UGAGGUAGUAGGUUGUGUGGUU
>mmu-let-7c-5p MIMAT0000523
(SEQ ID NO: 12)
UGAGGUAGUAGGUUGUAUGGUU
>mmu-let-7d-5p MIMAT0000383
(SEQ ID NO: 13)
AGAGGUAGUAGGUUGCAUAGUU
>mmu-let-7e-5p MIMAT0000524
(SEQ ID NO: 14)
UGAGGUAGGAGGUUGUAUAGUU
>mmu-let-7f-5p MIMAT0000525
(SEQ ID NO: 15)
UGAGGUAGUAGAUUGUAUAGUU
>mmu-let-7g-5p MIMAT0000121
(SEQ ID NO: 16)
UGAGGUAGUAGUUUGUACAGUU
>mmu-let-7i-5p MIMAT0000122
(SEQ ID NO: 17)
UGAGGUAGUAGUUUGUGCUGUU
>mmu-miR-98-5p MIMAT0000545
(SEQ ID NO: 18)
UGAGGUAGUAAGUUGUAUUGUU

Modulate Expression of Let7

Let7 expression can be modulated via various techniques available to an art worker. For example, it can be knocked down (through mutation for example) or knocked out/deleted. Further, silencing RNA, such as miRNA, shRNA, RNAi etc. can be used to decrease expression of the let7. Further inhibitory proteins can be used to downregulate the expression and/or activity of let7 (e.g., Lin-28). To increase its expression, vector expressing let7 can be introduced into a cell (transient or stable transfection/transduction), such as a T cell, or a strong promoter can be inserted in front the let7 coding sequence. Methods are further discussed herein below.

Also, LIN28 expression is reciprocal to that of mature let-7 (Viswanathan S. R. et al. (2008) Science 320 (5872): 97-100). LIN28 selectively binds the primary and precursor forms of let-7 and inhibits the processing of pri-let-7 to form the hairpin precursor (Newman M. A. et al. (2008) RNA 14: 1539-49). This binding is facilitated by the conserved loop sequence of primary let-7 family members and RNA-binding domains of LIN28 proteins (Piskounova E. et al. (2008). J. Biol. Chem. 283: 21310-21314). Thus, alteration of the expression of LIN28, such as through knock down (through mutation for example) or knock out/deleted methods can be used to modulate the expression of let7. Also, an antibody can be used to bind to LIN28 and decrease/inhibit its activity. To increase its expression, a vector expressing LIN28 can be introduced into a cell (transient or stable transfection/transduction), such as a T cell, or a strong promoter can be inserted in front the LIN28 coding sequence. Methods are further discussed herein below.

Also, expression of let-7 members can be controlled by MYC binding to their promoters. Therefore, let-7 expression can be modulated by modulating MYC expression.

As used herein, “inhibit” refers to a reduction (e.g., about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%) in the activity of let 7 as compared to the activity of let7 in the absence of an expression modifier.

Cancer/Autoimmune/Infectious Disorders/Diseases

Just as miRNA is involved in the normal functioning of eukaryotic cells, dysregulation of miRNA is associated with disease. The first human disease known to be associated with miRNA deregulation was chronic lymphocytic leukemia.

One embodiment provided herein is a method to treat cancer, autoimmune or infectious disorders/diseases, by administering to a subject in need thereof a composition that modulates the expression of let7, or cells that have been altered to have let expression modulated.

Cancer

Cancer is a group of diseases involving abnormal cell growth with the potential to invade or spread to other parts of the body. Not all tumors are cancerous; benign tumors do not spread to other parts of the body. Possible signs and symptoms include a lump, abnormal bleeding, prolonged cough, unexplained weight loss and a change in bowel movements. Over 100 cancers affect humans.

A number of cancers are recognized, including but not limited to, Bladder cancer, Lung cancer, Brain cancer, Melanoma, Breast cancer, Non-Hodgkin lymphoma, Cervical cancer, Ovarian cancer, Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer in Adolescents, Cancer in Children, Cancer in Young Adults, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Leukemia—Acute Lymphocytic (ALL) in Adults, Leukemia—Acute Myeloid (AML), Leukemia—Chronic Lymphocytic (CLL), Leukemia—Chronic Myeloid (CML), Leukemia—Chronic Myelomonocytic (CMML), Leukemia in Children, Liver Cancer, Lung Cancer, Lung Cancer—Non-Small Cell, Lung Cancer—Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.

Treating Cancer

Disclosed herein are methods of treating cancer.

Adoptive Cell Transfer

Adoptive cell transfer (ACT) is the transfer of cells into a patient. There are three forms of ACT that are used in cancer therapy: tumor infiltrating lymphocytes (TILs), T cells that are engineered to express anti-tumor T cell receptors (TCRs) and T cells with chimeric antigen receptors (CARs) (1). A side effect of CAR T cell therapy is toxicity caused by systemic production of inflammatory cytokines due to rapid death of large amount of tumor cells. Another challenge of ACT is that engineered T cells, like any other T cells including TILs, acquire exhausted phenotype due to immunosuppressive tumor microenvironment. It has been shown that less differentiated T cells with stem cell memory or central memory phenotype demonstrate better anti-tumor response in comparison to more differentiated effector T cells (2). Thus, generation of TCR T cells or CAR T cells that possess the features of memory cells, will minimize exhaustion and terminal differentiation of the infused T cells.

The cells may have originated from the patient him- or herself and then been altered before being transferred back, or, they may have come from another individual. The cells are most commonly derived from the immune system, with the goal of transferring improved immune functionality and characteristics along with the cells back to the patient. For example, T cells can be isolated, or differentiated from less mature cells, their let7 expression can be modulated and then the T cells/cells with modulated let7 expression can be placed in the patient.

### Genetically Modified Cells and Methods for Genetically Modifying Cells

T cells can be isolated or differentiated from less mature cells. They can then be genetically modified ex vivo. For example, a subject's T cells are isolated. The cells are then genetically altered to increase or decrease let7 expression. The cells can then be screened or selected ex vivo to identify those cells which have been successfully altered, and these cells can be introduced into the subject, either locally or systemically. The cells can then provide a stably-transfected source of cells that can express the desired level of let7. Especially where the patient's own cells are the source of the cells, this method provides an immunologically safe method for producing cells for transplant.

Cells isolated by the methods described herein can be genetically modified by introducing DNA or RNA into the cell by a variety of methods available to those of skill in the art. These methods are generally grouped into four major categories: (1) viral transfer, including the use of DNA or RNA viral vectors, such as retroviruses, including lentiviruses (Mochizuki, H., et al., 1998; Martin, F., et al. 1999; Robbins, et al. 1997; Salmons, B. and Gunzburg, W. H., 1993; Sutton, R., et al., 1998; Kafri, T., et al., 1999; Dull, T., et al., 1998), Simian virus 40 (SV40), adenovirus (see, for example, Davidson, B. L., et al., 1993; Wagner, E., et al., 1992; Wold, W., Adenovirus Methods and Protocols, Humana Methods in Molecular Medicine (1998), Blackwell Science, Ltd.; Molin, M., et al., 1998; Douglas, J., et al., 1999; Hofmann, C., et al., 1999; Schwarzenberger, P., et al., 1997), alpha virus, including Sindbis virus (U.S. Pat. No. 5,843,723; Xiong, C., et al., 1989; Bredenbeek, P. J., et al., 1993; Frolov, I., et al., 1996), herpes virus (Laquerre, S., et al., 1998) and bovine papillomavirus, for example; (2) chemical transfer, including calcium phosphate transfection and DEAE dextran transfection methods; (3) membrane fusion transfer, using DNA-loaded membranous vesicles such as liposomes (Loeffler, J. and Behr, J., 1993), red blood cell ghosts and protoplasts, for example; and (4) physical transfer techniques, such as microinjection, microprojectile J. Wolff in “Gene Therapeutics” (1994) at page 195. (see J. Wolff in “Gene Therapeutics” (1994) at page 195; Johnston, S. A., et al., 1993; Williams, R. S., et al., 1991; Yang, N. S., et al., 1990), electroporation, nucleofection or direct “naked” DNA transfer.

Cells can be genetically altered by insertion of pre-selected isolated DNA, by substitution of a segment of the cellular genome with pre-selected isolated DNA, or by deletion of or inactivation of at least a portion of the cellular genome of the cell. Deletion or inactivation of at least a portion of the cellular genome can be accomplished by a variety of means, including but not limited to genetic recombination, by antisense technology (which can include the use of peptide nucleic acids or PNAs), or by ribozyme technology, for example. Insertion of one or more pre-selected DNA sequences can be accomplished by homologous recombination or by viral integration into the host cell genome. Methods of non-homologous recombination are also known, for example, as described in U.S. Pat. Nos. 6,623,958, 6,602,686, 6,541,221, 6,524,824, 6,524,818, 6,410,266, 6,361,972, the contents of which are specifically incorporated by reference for their entire disclosure relating to methods of non-homologous recombination.

The desired gene sequence can also be incorporated into the cell, particularly into its nucleus, using a plasmid expression vector and a nuclear localization sequence. Methods for directing polynucleotides to the nucleus have been described in the art. For example, signal peptides can be attached to plasmid DNA, as described by Sebestyen, et al. (1998), to direct the DNA to the nucleus for more efficient expression.

The genetic material can be introduced using promoters that will allow for the gene of interest to be positively or negatively induced using certain chemicals/drugs, to be eliminated following administration of a given drug/chemical or can be tagged to allow induction by chemicals (including but not limited to the tamoxifen responsive mutated estrogen receptor) in specific cell compartments (including, but not limited to, the cell membrane).

Any of transfection or transduction technique can also be applied to introduce a transcriptional regulatory sequence into cells to activate a desired endogenous gene. This can be done by both homologous (e.g., U.S. Pat. No. 5,641,670) or non-homologous (e.g., U.S. Pat. No. 6,602,686) recombination. These patents are incorporated by reference for teaching of methods of endogenous gene activation.

Successful transfection or transduction of target cells can be demonstrated using genetic markers, in a technique that is known to those of skill in the art. The green fluorescent protein of Aequorea victoria, for example, has been shown to be an effective marker for identifying and tracking genetically modified hematopoietic cells (Persons, D., et al., 1998). Alternative selectable markers include the 13-Gal gene, the truncated nerve growth factor receptor, drug selectable markers (including but not limited to NEO, MTX, hygromycin).

Formulations, Dosage Forms and Routes of Administration

Cells can be administered systemically or locally. The route of administration used can depend upon the disease/disorder being treated or prevented.

For the purposes described herein, either autologous, allogeneic or xenogeneic cells, or their differentiated progeny, can be administered to a subject, either in differentiated or undifferentiated form, genetically altered or unaltered, by direct injection to a tissue site, systemically, on a surface, on or around the surface of an acceptable matrix, encapsulated or in combination with a pharmaceutically acceptable carrier.

The cells can be provided in a pharmaceutical composition. The pharmaceutical composition can comprise pharmaceutically acceptable diluent(s), excipient(s), or carrier(s). The pharmaceutical compositions can include other medicinal or pharmaceutical agents, carriers, adjuvants, such as preserving, stabilizing, wetting or emulsifying agents, solution promoters, salts for regulating the osmotic pressure, and/or buffers.

Methods well known in the art for making formulations are to be found in, for example, Remington: The Science and Practice of Pharmacy, (20th ed.) ed. A. R. Gennaro A R., 2000, Lippencott Williams & Wilkins. Formulations for parenteral administration may, for example, contain as excipients sterile water or saline, polyalkylene glycols such as polyethylene glycol, oils of vegetable origin, or hydrogenated naphthalenes, biocompatible, biodegradable lactide polymer, or polyoxyethylene-polyoxypropylene copolymers may be used to control the release of the present factors. Other potentially useful parenteral delivery systems for the factors include ethylene-vinyl acetate copolymer particles, osmotic pumps, implantable infusion systems, and liposomes. Formulations for inhalation may contain as excipients, for example, lactose, or may be aqueous solutions containing, for example, polyoxyethylene-9-lauryl ether, glycocholate and deoxycholate, or may be oily solutions for administration in the form of nasal drops, or as a gel to be applied intranasally.

The appropriate dosage of cells will depend, for example, on the condition to be treated, the severity and course of the condition, whether the cells are administered for preventive or therapeutic purposes, previous therapy, the patient's clinical history and response to cells, the type of cells used, and the discretion of the attending physician. Cells are suitably administered to the patient at one time or over a series of treatments and may be administered to the patient at any time as necessary for treatment or prevention of disease/disorder. Cells may be administered as the sole treatment or in conjunction with other drugs or therapies useful in treating the condition in question.

The quantity of cells to be administered will vary for the subject being treated. In one embodiment, between about 10⁴ to about 10⁸, such as about 10⁵ to about 10′ and including, about 3×10⁷ stem cells can be administered to a human subject. However, the precise determination of what would be considered an effective dose may be based on factors individual to each patient, including their size, age, disease or injury, amount of damage, amount of time since the damage occurred and factors associated with the mode of delivery (direct injection—lower doses, intravenous—higher doses). Dosages can be readily ascertained by those skilled in the art from this disclosure and the knowledge in the art.

Preferable ranges of purity in populations comprising cells, or their differentiated progeny, are about 50-55%, about 55-60%, and about 65-70%. In some embodiments, the purity is about 70-75%, about 75-80%, about 80-85%; and including the purity of about 85-90%, about 90-95%, and about 95-100%. However, populations with lower purity can also be useful, such as about 25-30%, about 30-35%, about 35-40%, about 40-45% and about 45-50%. Purity of cells can be determined according to the gene expression profile within a population.

The skilled artisan can readily determine the number of cells and optional additives, vehicles, or carrier in compositions to be administered in methods of the invention. Typically, additives (in addition to the cell(s) and/or cytokine(s)) are present in an amount of about 0.001 to about 50 wt % solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, and about 0.05 to about 5 wt %. Of course, for any composition to be administered to an animal or human, and for any particular method of administration, it is practical to determine therefore: toxicity, such as by determining the lethal dose (LD) and LD₅₀ in a suitable animal model e.g., a rodent, such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. Additionally, the time for sequential administrations can be ascertained without undue experimentation.

When administering a therapeutic composition of the present invention, it will generally be formulated in a unit dosage injectable form (solution, suspension, emulsion). The pharmaceutical formulations suitable for injection include sterile aqueous solutions and dispersions. The carrier can be a solvent or dispersing medium containing, for example, water, saline, phosphate buffered saline, polyol (for example, glycerol, propylene glycol, liquid polyethylene glycol, and the like) and suitable mixtures thereof.

Additionally, various additives which enhance the stability, sterility, and isotonicity of the compositions, including antimicrobial preservatives, antioxidants, chelating agents and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the cells.

Sterile injectable solutions can be prepared by incorporating the cells utilized in practicing the present invention in the required amount of the appropriate solvent with various amounts of the other ingredients, as desired.

In one embodiment, cells can be administered initially, and thereafter maintained by further administration of cells. For instance, cells can be administered by one method of injection, and thereafter further administered by a different or the same type of method.

Examples of compositions comprising cells include liquid preparations for administration, including suspensions, and, preparations for direct or intravenous administration (e.g., injectable administration), such as sterile suspensions or emulsions. Such compositions may be in admixture with a suitable carrier, diluent, or excipient such as sterile water, physiological saline, glucose, dextrose, or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation.

Compositions are conveniently provided as liquid preparations, e.g., isotonic aqueous solutions, suspensions, emulsions or viscous compositions, which may be buffered to a selected pH. Liquid preparations are normally easier to prepare than gels, other viscous compositions and solid compositions. Additionally, liquid compositions are somewhat more convenient to administer, especially by injection. Viscous compositions, on the other hand, can be formulated within the appropriate viscosity range to provide longer contact periods with specific tissues.

The choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Solutions, suspensions and gels normally contain a major amount of water (preferably purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose), may also be present. The compositions can be isotonic, i.e., they can have the same osmotic pressure as blood and lacrimal fluid.

The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is one option for buffers containing sodium ions.

Viscosity of the compositions, if desired, can be maintained at the selected level using a pharmaceutically acceptable thickening agent. Methylcellulose can be used as it is readily and economically available and is easy to work with. Other suitable thickening agents include, for example, xanthan gum, carboxymethyl cellulose, hydroxypropyl cellulose, carbomer, and the like. The concentration of the thickener will depend upon the agent selected and the desired viscosity. Viscous compositions are normally prepared from solutions by the addition of such thickening agents.

A pharmaceutically acceptable preservative or cell stabilizer can be employed to increase the life of the compositions. If preservatives are necessary, it is well within the purview of the skilled artisan to select compositions that will not affect the viability or efficacy of the cells described in the present invention.

Those skilled in the art will recognize that the components of the compositions should be selected to be chemically inert. This will present no problem to those skilled in chemical and pharmaceutical principles, or problems can be readily avoided by reference to standard texts or simple experiments (not involving undue experimentation), from this disclosure and the documents cited herein.

Suitable regimes for initial administration and further doses or for sequential administrations also are variable, may include an initial administration followed by subsequent administrations; but nonetheless, can be ascertained by the skilled artisan, from this disclosure, the documents cited herein, and the knowledge in the art.

Combined Treatment

The cells and composition of the invention can be combined with other, including current, therapies to treat the cancer, autoimmune disease or infectious disease. For example, cells and composition of the invention can be combined with other treatments, including other cancer treatments, such as chemotherapy, radiation and/or other immunotherapies, such as TIL, TCR, CAR T therapy, dendritic cell-based pump-priming. T-cell adoptive transfer, immune enhancement therapy, genetically engineered T cells, immune recovery and/or vaccination.

### EXAMPLE

The following example is provided in order to demonstrate and further illustrate certain embodiments and aspects of the present invention and are not to be construed as limiting the scope thereof.

### Example 1—Let-7 Promotes Superior Anti-Tumor Activity of CD8 T Cells and Memory Formation

It was previously published that let-7 miRNA family controls the homeostasis of naive CD8 T cells and their differentiation into effector cytotoxic T lymphocytes (CTLs). In particular, it was shown that let-7 deficient CTLs (Lin28Tg) expressed high levels of cytolytic proteins (Granzyme A, Granzyme B and Perforin) and demonstrated a high in vitro cytotoxic activity in comparison to WT CTLs. On the contrary, in vitro Let-7 overexpression (Let7Tg) greatly suppressed the differentiation of CD8 T cells into functional effectors. Based on these data it was predicted that let-7 deficient CTLs will have enhanced effector functions in vivo.

Subcutaneous B16 Melanoma Model

The prediction was tested by using a well-established mouse tumor model of subcutaneous (s.c.) B16F10 melanoma with adoptive transfer of melanoma-specific CTLs. To eliminate bystander effects from other T cells on the development and differentiation/function of CTLs and to focus CTL responses on a single antigen, two double transgenic mouse strains P14+Lin28Tg and P14+Let-7Tg on RAG2-knockout background (where P14 is the CD8-specific T cell receptor that recognizes a specific antigen, the LCMV-derived gp33 peptide) were generated. To ensure antigen-specific tumor recognition by P14 CTLs, the B16F10 melanoma cell line was transduced with a mini-gene encoding the gp33 peptide and a GFP reporter (gp33-IRES-GFP).

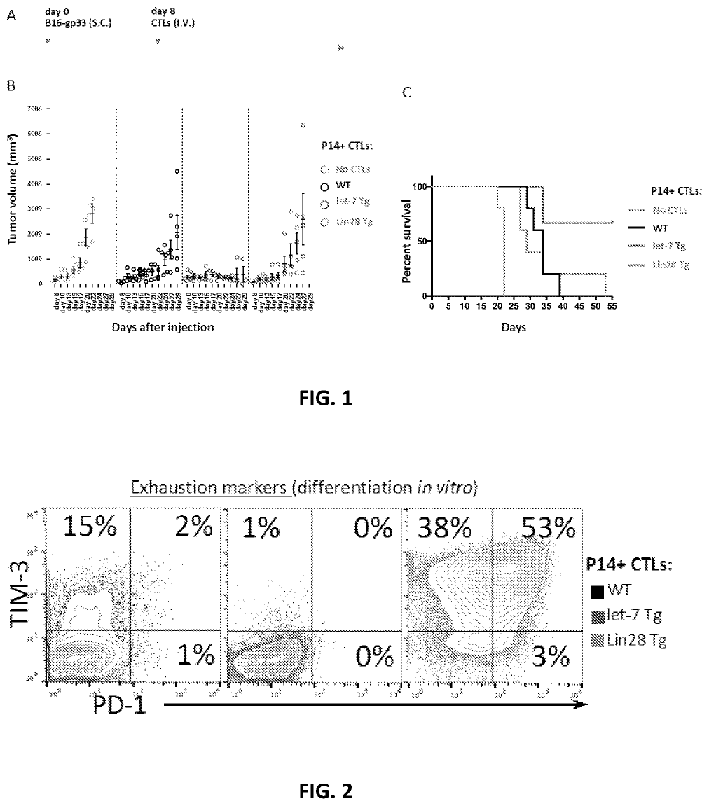

C57BL/6 mice were sub-lethally irradiated (500 Rad) and injected s.c. in the flank with 2.5×10⁵ tumor cells/mouse. 8 days later when the tumors became visible, in vitro differentiated P14+ CTLs (1.5×10⁶ cells/mouse) were adoptively transferred into mice. Four experimental groups with n=5 were created: no CTL injection, P14+WT CTLs, P14+Let-7Tg CTLs and P14+Lin28Tg CTLs. Tumors were measured every 2-3 days with a caliper (FIG. 1A). Tumor volume was determined using the following formula: V=0.5×L×W², where L is tumor length and W is tumor width.

Mice that didn't receive CTLs had to be sacrificed on day 22 due to large tumor volume. As expected, P14+WT CTLs controlled tumor growth at the beginning, but eventually tumors escaped, and mice had to be sacrificed on day 29. Surprisingly, results with let-7 deficient CTLs turned out to be the opposite to what was originally predicted. P14+Lin28Tg CTLs demonstrated a very poor cytotoxic activity with tumors growing larger and faster than in mice with WT CTLs. On the contrary, P14+Let-7Tg CTLs controlled tumor growth very well, where most of the tumors started to shrink and eventually disappeared by the end of the experiment (FIGS. 1B and C). Similar results were obtained using a different tumor model (thymoma EL-4).

Thus, in spite of an outstanding cytotoxic function in vitro, let-7 deficient CTLs failed to control tumor growth in vivo. Instead, let-7 transgenic CTLs which had decreased expression of cytolytic proteins and demonstrated a lower killing activity towards specific targets in vitro, turned out to be fully functional in vivo and managed to inhibit melanoma growth in subcutaneous model.

Molecular Basis for the Role of Let-7 in CTL Differentiation and Tumor Control

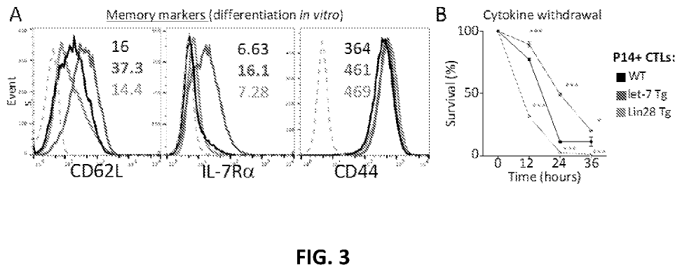

In order to deeper understand the underlying molecular mechanisms of the let-7 mediated phenotype, RNASeq analysis of day 5 in vitro differentiated CTLs from P14+WT, P14+Let-7Tg and P14+Lin28Tg mice was performed. Differential gene expression analysis confirmed that P14+Lin28Tg CTLs have increased expression of activation markers and cytolytic molecules. In addition, surprisingly, let-7 deficient CTLs had a clear “exhaustion signature” with most of the inhibitory receptors including PD1, Lag3, Tim3, CD160, and 2B4 being upregulated. In contrast, Let-7Tg CTLs possessed a “memory signature” with such genes as CD62L, IL-7Ra, TCF7, CCR7, LEF1 and ID3 being upregulated. RNAseq data was confirmed by surface staining and flow cytometry analysis and it was found that indeed let-7 deficient CTLs up-regulated PD-1, Tim-3 (FIG. 2), Lag3, CD160 and 2B4 (not shown) receptors 5 days after differentiation culture in vitro, while let-7 transgenic CTLs completely lacked their expression. At the same time let-7Tg CTLs expressed memory markers such as CD62L and IL-7Ra at much higher level than WT or Lin28Tg CTLs, while being equally activated based on the expression of CD44 (FIG. 3A). Furthermore, survival rate upon IL-2 withdrawal was significantly higher for let-7Tg CTLs which is another well-known feature of memory cells (FIG. 3B).

Thus, the in vivo data support the conclusion that let-7 will prevent exhaustion and promote the differentiation of CD8 cells into memory population, while the absence of let-7 will result in the generation of exhausted non-functional CTLs within tumor microenvironment in vivo.

Pulmonary Metastasis B16 Melanoma Model

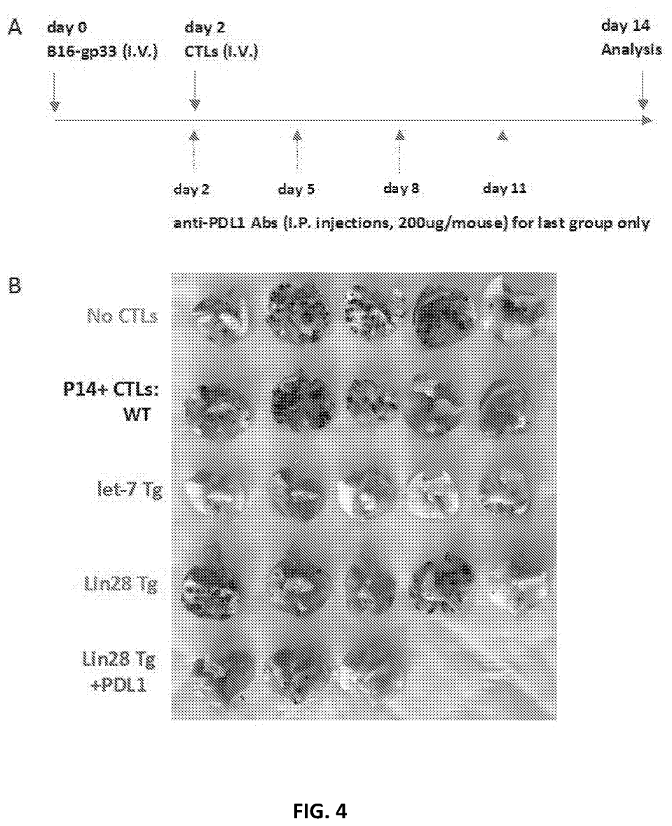

To confirm these results, a pulmonary metastasis model of B16 melanoma was used. For this experiment, mice were injected i.v. with 2×10⁵ tumor cells/mouse and then 2 days later with in vitro differentiated P14+ CTLs (2×10⁶ cells/mouse). Five experimental groups were created: no CTL injection, P14+WT CTLs, P14+Let-7Tg CTLs and P14+Lin28Tg CTLs with or without anti-PDL1 checkpoint blockade therapy, where mice received 4 i.p. injections of 200 ug/mouse of anti-PDL1 antibody on day 2, 5, 8 and 11. On day 14 mice were euthanized and lung metastasis were counted (FIG. 4A).

Mice that did not receive any CTLs had numerous metastasis on the surface of the lungs. Both P14+WT and P14+Lin28Tg CTLs controlled the growth of metastasis to a certain extent. Anti-PDL1 therapy greatly improved the outcome of P14+Lin28Tg CTL treatment but did not completely protect mice from the development of metastasis. Lungs from all 5 mice that received P14+Let-7Tg CTLs appeared to be absolutely clean and had no visible metastasis (FIG. 4B).

The data confirms that (i) without let-7 CD8 T cells become “over-differentiated” and easily exhausted within suppressive tumor microenvironment and (ii) let-7 expression is necessary for the generation of long-lived memory cells capable of protection against tumor development.

Summary

It was determined that miRNA (let-7) regulates T cell responses (including both T-helpers and Cytotoxic CD8+T Lymphocytes (CTLs)). In particular, it was shown that let-7 deficient CTLs demonstrated an incredibly high cytotoxic activity in vitro in comparison to WT CTLs, while let-7 overexpression greatly suppressed the differentiation of CD8 T cells into functional effectors in vitro. However, in vivo experiments demonstrated that maintaining high let-7 expression levels in CTLs results in dramatic increase in anti-tumor activity of these cells, in spite of their poor performance in cytotoxic assays in vitro. Conversely, it was found that when let-7 expression levels are low in CTLs, they have diminished ability to respond in vivo regardless of their outstanding cytotoxic function in vitro. Transcriptome analysis of let-7 deficient and let-7 overexpressing cells revealed that let-7 promotes memory formation and inhibits exhaustion of T cells. Thus, (i) let-7 overexpression will result in the generation of memory T cells and (ii) delivering let-7 miRNA or increasing its expression in adoptive cell therapies will enhance the CTL anti-tumor response when these cells are injected back into patients.

All three types of ACT will benefit from increasing let-7 expression, including, for example, CAR T cell therapy. By introducing let-7 into engineered T cells two goals will be achieved: (i) since let-7 expressing CTLs are less efficient killers, tumors will not be destroyed as fast as they are in the case of regular CAR T cells and therefore, toxicity will be reduced and (ii) since let-7 overexpression leads to the acquisition of memory phenotype, engineered T cells will not become exhausted, will be long-lived and will ensure complete tumor clearance.

### BIBLIOGRAPHY

1. June C H, O'Connor R S, Kawalekar O U, Ghassemi S, Milone M C. CAR T cell immunotherapy for human cancer. Science. 2018 Mar. 23; 359(6382):1361-1365
2. Klebanoff C A, Gattinoni L, Restifo N P. Sorting through subsets: which T-cell populations mediate highly effective adoptive immunotherapy? J Immunother. 2012; 35(9):651-60. Wells A C et al, Modulation of let-7 miRNAs controls the differentiation of effector CD8 T cells. Elife. 2017 Jul. 24; 6. pii: e26398. doi: 10.7554/eLife.26398.
Pobezinsky L A, Wells A C. Let's fight cancer: let-7 is a tool to enhance antitumor immune responses. Future Oncol. 2018 May; 14(12):1141-1145. doi: 10.2217/fon-2018-0037
PCT/US2017/030657

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. A composition comprising lymphocytes or T cells with increased expression of let7 as compared to wild-type cells, wherein the lymphocytes or T cells comprise decreased expression of markers PD1, Lag3, Tim3, CD160 and 2B4 and increased expression of markers CD62L, IL-7Ra, TCF7, CCR7, LEF1 and ID3 as compared to wild type T cells.

2. A method to treat cancer comprising administering to a subject in need thereof an effective amount of a composition of lymphocytes or T cells with increased expression of let7 as compared to wild type cells T cells.

3. The method of claim 2, wherein the lymphocytes or T cells further comprise decreased expression of markers PD1, Lag3, Tim3, CD160 and 2B4 and increased expression of markers CD62L, IL-7Ra, TCF7, CCR7, LEF1 and ID3 as compared to wild type T cells.

4. The composition of claim 1, wherein the T cells are cytotoxic T lymphocyte cells.

5. The composition of claim 1, wherein the T cells are engineered to express anti-tumor T cell receptors (TCRs) or are T cells with chimeric antigen receptors (CARs).

6. The composition of claim 1, wherein the lymphocytes are tumor infiltrating lymphocytes (TILs).

7. The method of claim 2, wherein the cancer is melanoma or lymphoma.

8. A method to increase cytotoxic activity of cytotoxic T lymphocyte (CTL) cells in vivo comprising increasing the level of let7 expression in said CTL cells.

9. The method of claim 2, wherein the let7 expression is increased by increasing let7 copy number or by increasing expression by inserting a strong promoter in front of let7 coding region.

10. The method of claim 9, further comprising administering at least one or more additional cancer treatments.

11. The method of claim 10, where the one or more additional treatments comprise chemotherapy, radiation and/or immunotherapy.


SEQUENCE LISTING (18 sequences)

SEQ ID NO: 1 - RNA, 22 nt, Homo sapiens
ugagguaguagguuguauaguu

SEQ ID NO: 2 - RNA, 22 nt, Homo sapiens
ugagguaguagguugugugguu

SEQ ID NO: 3 - RNA, 22 nt, Homo sapiens
ugagguaguagguuguaugguu

SEQ ID NO: 4 - RNA, 22 nt, Homo sapiens
agagguaguagguugcauaguu

SEQ ID NO: 5 - RNA, 22 nt, Homo sapiens
ugagguaggagguuguauaguu

SEQ ID NO: 6 - RNA, 22 nt, Homo sapiens
ugagguaguagauuguauaguu

SEQ ID NO: 7 - RNA, 22 nt, Homo sapiens
ugagguaguaguuuguacaguu

SEQ ID NO: 8 - RNA, 22 nt, Homo sapiens
ugagguaguaguuugugcuguu

SEQ ID NO: 9 - RNA, 22 nt, Homo sapiens
ugagguaguaaguuguauuguu

SEQ ID NO: 10 - RNA, 22 nt, Mus musculus
ugagguaguagguuguauaguu

SEQ ID NO: 11 - RNA, 22 nt, Mus musculus
ugagguaguagguugugugguu

SEQ ID NO: 12 - RNA, 22 nt, Mus musculus
ugagguaguagguuguaugguu

SEQ ID NO: 13 - RNA, 22 nt, Mus musculus
agagguaguagguugcauaguu

SEQ ID NO: 14 - RNA, 22 nt, Mus musculus
ugagguaguagguuguaugguu

SEQ ID NO: 15 - RNA, 22 nt, Mus musculus
ugagguaguagauuguauaguu

SEQ ID NO: 16 - RNA, 22 nt, Mus musculus
ugagguaguaguuuguacaguu

SEQ ID NO: 17 - RNA, 22 nt, Mus musculus
ugagguaguaguuugugcuguu

SEQ ID NO: 18 - RNA, 22 nt, Mus musculus
ugagguaguaaguuguauuguu